(19) United States
(12) Patent Application Publication
(10) Pub. No.: US 2024/0374906 A1
(43) Pub. Date: Nov. 14, 2024

(54) THERMOPLASTIC BACKFILL FOR SEALED ELECTRONICS

(71) Applicant: Cardiac Pacemakers, Inc., St. Paul, MN (US)

(72) Inventors: Joseph Thomas Delaney, JR., Minneapolis, MN (US); James Michael English, Cahir (IE)

(21) Appl. No.: 18/657,662

(22) Filed: May 7, 2024

Related U.S. Application Data
(60) Provisional application No. 63/501,583, filed on May 11, 2023.

(51) Int. Cl. A61N 1/375; C08L 25/08; C08L 53/00; C08L 91/00

(52) U.S. Cl. CPC A61N 1/3758; C08L 25/08; C08L 53/00; C08L 91/00; C08L 2203/02

(57) ABSTRACT

A method of manufacturing an implantable medical device having electrical components disposed within a cavity of a housing. The method comprises heating a gel filler material to a liquid, flowable state, depositing the gel filler material in the liquid, flowable state into the cavity to substantially encapsulate the electrical components within the gel filler material; and allowing the gel filler material to cool to a semi-solid state.

### CROSS REFERENCE TO RELATED APPLICATION

The present application claims priority to U.S. Provisional Patent Application Ser. No. 63/501,583, filed May 11, 2023, the entire disclosure of which is incorporated herein by reference.

### TECHNICAL FIELD

The present disclosure relates to medical device, and more particularly, to implantable medical devices having internal electrical components inside a housing.

### BACKGROUND

Some medical devices include a housing that holds and protects electronic components. By way of example, implantable cardiac rhythm management (CRM) devices such as pacemakers and implantable cardioverter defibrillators frequently include a housing that hermetically seals off an interior volume. Components, such as electronic components, used to generate and control electrical stimulation pulses are then disposed within the hermetically sealed interior volume of the housing.

Frequently, there is some amount of the volume of the housing that remains unused after the device components are placed within the housing. This space can be referred as the “residual volume” or the “free interior volume”. In some circumstances, this residual volume has simply been filled with ambient air. In other circumstances, the residual volume of housings has been filled with a non-reactive gas such as pure nitrogen. In still other circumstances, the residual volume of housings has been filled with a solid such as an epoxy.

### SUMMARY

In Example 1, a method of manufacturing an implantable medical device having electrical components disposed within a cavity of a housing. The method comprises heating a gel filler material to a liquid, flowable state, depositing the gel filler material in the liquid, flowable state into the cavity to substantially encapsulate the electrical components within the gel filler material, and allowing the gel filler material to cool to a semi-solid state.

In Example 2, the method of Example 1, wherein the gel filler material comprises a styrene-ethylene-butylene-styrene (SEBS) thermoplastic block copolymer and a plasticizing oil.

In Example 3, the method of Example 2, wherein the housing comprises a first housing part and the cavity comprises a first cavity portion defined by the first housing part, and wherein a first set of the electrical components is disposed within the first cavity portion, and wherein depositing the gel filler material includes depositing the gel filler material in the first cavity portion to substantially encapsulate the first set of the electrical components.

In Example 4, the method of Example 3, wherein the housing comprises a second housing part and the cavity comprises a second cavity portion defined by the second housing part, and wherein a second set of the electrical components is disposed within the second cavity portion, and wherein depositing the gel filler material includes depositing the gel filler material in the second cavity portion to substantially encapsulate the second set of the electrical components.

In Example 5, the method of Example 4, further comprising joining the first and second housing parts after allowing the gel filler material to cool to the substantially semi-solid state.

In Example 6, the method of Example 2, further comprising prior to depositing the gel filler material, forming the housing by joining first and second housing parts to define the cavity with the electrical components disposed therein, wherein the housing includes a fill port and an exhaust port; and wherein depositing the gel filler material includes introducing the gel filler material into the cavity through the fill port, and hermetically sealing the fill port and the exhaust port after allowing the gel filler material to cool to the substantially solid state.

In Example 7, the method of Example 2, wherein heating the gel filler material includes heating the gel filler material to a temperature above 70 degrees C.

In Example 8, the method of Example 8, wherein allowing the gel filler material to cool includes allowing the gel filler to cool to a temperature below 70 degrees C.

In Example 9, the method of Example 8, wherein allowing the gel filler material to cool includes actively cooling the gel filler material in an environment below ambient temperature.

In Example 10, the method of Example 2, wherein the plasticizing oil is a mineral oil.

In Example 11, an implantable medical device, comprising a housing defining a hermetically sealed cavity, electrical components rigidly mounted within the housing, and a semi-solid gel filler material disposed within the cavity and substantially encapsulating the electrical components, the gel filler material comprising a thermoplastic block copolymer and a plasticizing mineral oil.

In Example 12, the implantable medical device of Example 11, wherein the thermoplastic block copolymer is a styrene-ethylene-butylene-styrene (SEBS) thermoplastic block copolymer.

In Example 13, the implantable medical device of Example 12, wherein the housing comprises a first housing part and the cavity comprises a first cavity portion defined by the first housing part, and wherein a first set of the electrical components is disposed within the first cavity portion, and wherein the semi-solid gel filler material is disposed in the first cavity portion to substantially encapsulate the first set of the electrical components.

In Example 14, the implantable medical device of Example 13, wherein the housing comprises a second housing part and the cavity comprises a second cavity portion defined by the second housing part, and wherein a second set of the electrical components is disposed within the second cavity portion, and wherein the semi-solid gel filler material is disposed in the second cavity portion to substantially encapsulate the second set of the electrical components.

In Example 15, the implantable medical device of Example 14, wherein the first housing part and the second housing part are joined to form the housing.

In Example 16, a method of manufacturing an implantable medical device having electrical components disposed within a cavity of a housing, the method comprising heating a gel filler material to a liquid, flowable state, wherein the gel filler material comprises a styrene-ethylene-butylene-styrene (SEBS) thermoplastic block copolymer and a plasticizing oil, depositing the gel filler material in the liquid, flowable state into the cavity to substantially encapsulate the electrical components within the gel filler material, and allowing the gel filler material to cool to a semi-solid state.

In Example 17, the method of Example 16, wherein the housing comprises a first housing part and the cavity comprises a first cavity portion defined by the first housing part, and wherein a first set of the electrical components is disposed within the first cavity portion, and wherein depositing the gel filler material includes depositing the gel filler material in the first cavity portion to substantially encapsulate the first set of the electrical components.

In Example 18, the method of Example 17, wherein the housing comprises a second housing part and the cavity comprises a second cavity portion defined by the second housing part, and wherein a second set of the electrical components is disposed within the second cavity portion, and wherein depositing the gel filler material includes depositing the gel filler material in the second cavity portion to substantially encapsulate the second set of the electrical components.

In Example 19, the method of Example 18, further comprising joining the first and second housing parts after allowing the gel filler material to cool to the substantially semi-solid state.

In Example 20, the method of Example 16, further comprising prior to depositing the gel filler material, forming the housing by joining first and second housing parts to define the cavity with the electrical components disposed therein, wherein the housing includes a fill port and an exhaust port; and wherein depositing the gel filler material includes introducing the gel filler material into the cavity through the fill port, and hermetically sealing the fill port and the exhaust port after allowing the gel filler material to cool to the substantially solid state.

In Example 21, the method of Example 16, wherein heating the gel filler material includes heating the gel filler material to a temperature above 70 degrees C.

In Example 22, the method of Example 21, wherein allowing the gel filler material to cool includes allowing the gel filler to cool to a temperature below 70 degrees C.

In Example 23, the method of Example 21, wherein allowing the gel filler material to cool includes actively cooling the gel filler material in an environment below ambient temperature.

In Example 24, the method of Example 16, wherein the plasticizing oil is a mineral oil.

In Example 25, an implantable medical device, comprising a housing defining a hermetically sealed cavity, electrical components mounted within the housing, and a semi-solid gel filler material disposed within the cavity and substantially encapsulating the electrical components, the gel filler material comprising a thermoplastic block copolymer.

In Example 26, the implantable medical device of Example 25, wherein the thermoplastic block copolymer is a styrene-ethylene-butylene-styrene (SEBS) thermoplastic block copolymer.

In Example 27, the implantable medical device of Example 26, wherein the gel filler material further comprises a plasticizing oil.

In Example 28, the implantable medical device of Example 27, wherein the plasticizing oil is a mineral oil.

In Example 29, the implantable medical device of Example 28, wherein the housing comprises a first housing part and the cavity comprises a first cavity portion defined by the first housing part, and wherein a first set of the electrical components is disposed within the first cavity portion, and wherein the semi-solid gel filler material is disposed in the first cavity portion to substantially encapsulate the first set of the electrical components.

In Example 30, the implantable medical device of Example 29, wherein the housing comprises a second housing part and the cavity comprises a second cavity portion defined by the second housing part, and wherein a second set of the electrical components is disposed within the second cavity portion, and wherein the semi-solid gel filler material is disposed in the second cavity portion to substantially encapsulate the second set of the electrical components.

In Example 31, the implantable medical device of Example 30, wherein the first housing part and the second housing part are joined to form the housing.

In Example 32, an implantable medical device comprising a hermetically sealed housing forming an internal cavity, electrical components mounted within the cavity of the housing, wherein portions of the cavity not occupied by the electrical components define a residual volume of the housing, and a semi-solid gel filler material filling the residual volume and substantially encapsulating the electrical components, the gel filler material assuming a flowable liquid state at a temperature of 70 degrees Celsius or greater, and capable of transitioning to semi-solid state at a temperature below 70 degrees Celsius.

In Example 33, the implantable medical device of Example 32, wherein the semi-solid gel filler material comprises a thermoplastic block copolymer and a plasticizing oil.

In Example 34, the implantable medical device of Example 33, wherein the thermoplastic block copolymer is a styrene-ethylene-butylene-styrene (SEBS) thermoplastic block copolymer.

In Example 35, the implantable medical device of Example 34, wherein the plasticizing oil is a mineral oil.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic illustration of an exemplary implantable medical device (IMD) that can be used in relation to embodiments of the present disclosure.

FIG. 2 is a cross-sectional schematic view of the implantable medical device of FIG. 1, according to an embodiment of the present disclosure.

FIG. 3 is a flow chart illustrating an exemplary method of manufacturing the implantable medical device of FIG. 1, according to embodiments of the present disclosure.

FIG. 4 is a flow chart illustrating an exemplary alternative method of manufacturing the implantable medical device of FIG. 1, according to embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

Frequently, there is some volumetric amount of a medical device housing that remains unused after the device components are placed within the housing. This unused volume can be referred to as a “residual volume” or “free interior volume”. In some instances in the past, this residual volume has simply been filled with ambient air. However, ambient air contains components such as oxygen that can contribute to the degradation of some materials. In other circumstances, the residual volume of housings was filled with a non-reactive gas such as pure nitrogen. In still other circumstances, the residual volume of housings was filled with a material that forms a rigid solid such as an epoxy. However, a solid can undesirably serve to concentrate stresses inside the housing in certain areas.

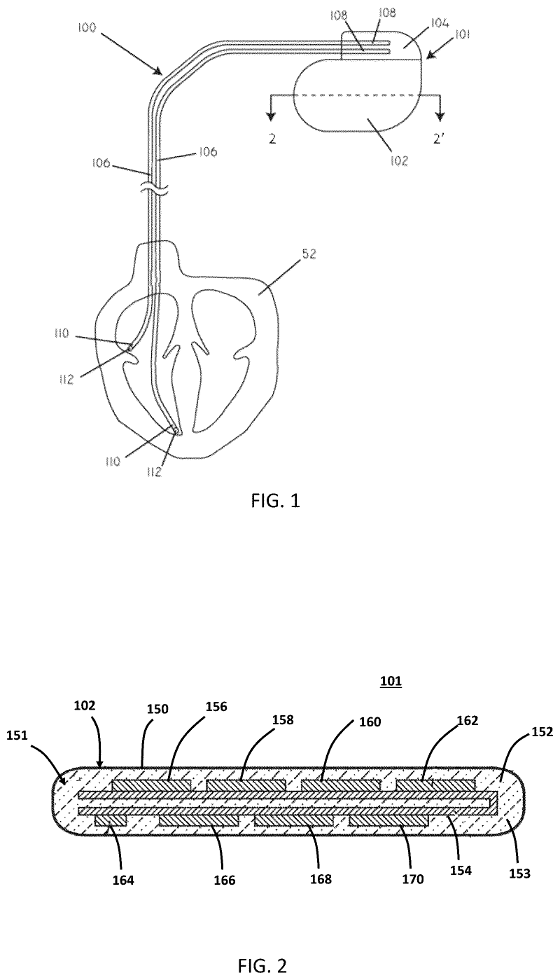

FIG. 1 is a schematic illustration of an implantable medical system 100 in accordance with an embodiment disposed within the heart 52 of a subject. The implantable medical system 100 includes a pulse generator 101 and one or more leads 106. Depending on the configuration, the leads 106 can provide electrical and/or optical communication between the distal ends of the leads 106 and the pulse generator 101. In FIG. 1, the distal ends of the leads 106 are located in the subject's heart 52. However, it will be appreciated that the distal ends of the leads 106 could be disposed in other locations.

In various embodiments, the implantable medical device 100 can include a cardiac rhythm management device, such as a pacemaker, a cardiac resynchronization therapy (CRT) device, a remodeling control therapy (RCT) device, a cardioverter/defibrillator, or a pacemaker-cardioverter/defibrillator. In some embodiments, the implantable medical device 101 can include a neurological stimulation device. It will be appreciated that embodiments of the disclosure can also be used in conjunction with implantable medical devices that lack pulse generators, but include a housing, such as monitoring devices and drug delivery devices.

As illustrated in FIG. 1, the pulse generator 101 can include a housing 102 that serves to hold and protect various components of the device, such as a component module or an electronics module. The housing 102 is coupled to a header module 104. The header module 104 can include ports to receive the proximal ends 108 of the leads 106. The header module can be made from various materials such as polymers. The distal ends 110 of the leads 106 can include electrodes 112 that can interface with tissue of the subject's heart 52. However, it will be appreciated that in some embodiments electrodes can be included at locations other than the distal ends of the leads.

FIG. 2 is a schematic illustration of a cross-section of the pulse generator 101 taken along line 2-2′ in FIG. 1. The housing 102 includes a housing wall 150. The housing wall 150 can be made of various materials including metals, polymers, and ceramics. In some embodiments, the housing wall 150 can be substantially rigid. In a particular embodiment, the housing wall 150 is made substantially or entirely of titanium or an alloy thereof. In the various embodiments, the housing 102 forms a hermetically sealed enclosure about various device components disposed therein, as explained in greater detail below.

As shown, the housing wall 150 forms a cavity defining an interior volume 151. Various device components can be disposed within the interior volume 151. For example, a circuit board 154 can be disposed within the interior volume 151. Various electrical components 156, 158, 160, 162, 164, 166, 168, 170 can be mounted on the circuit board 154 within the housing 102, or can be mounted to the housing wall 150 itself using various structural components (not shown in FIG. 2). The electrical components can include various items for operation of the device including microprocessors, batteries, capacitors, telemetry modules, amplifiers, transducers, converters, filters, various types of integrated circuit chips (IC chips), and the like. The residual volume 153 is the portion of the interior volume 151 not occupied by the above-mentioned electrical components and related structural elements.

In the illustrated embodiment, filler material 152 is disposed within the residual volume 153 to substantially or completely fill the residual volume 153 and partially or completely encapsulate some or all of the electrical components (e.g., the circuit board 154 and the electronic components 156, 158, 160, 162, 164, 166, 168 mounted thereon). It will be appreciated that the total volume of the filler material 152 used with embodiments herein can vary based on the size of the housing, the percentage of residual volume within the housing with respect to total volume, the degree to which the residual volume is filled, and the like.

In the various embodiments, the filler material 152 comprises a gel material that assumes a semi-solid state typical of organogel materials. The resulting organogel thermoplastic polymer that can be processed at relatively low temperatures to assume a flowable, liquid state when heated to a temperature below that which would adversely affect the electrical components disposed within the housing 102. In embodiments, the filler material 152 comprises thermoplastic block copolymer that is plasticized by blending with a plasticizing oil. In one embodiment, for example, a styrene-ethylene-butylene-styrene (SEBS) thermoplastic block copolymer plasticized with a plasticizing oil such as mineral oil or parrafin oil can assume a flowable state at temperatures as low as 70 degrees Celsius. The flowable state facilitates depositing the filler material 152 into the residual volume 153, which due to the low-temperature processing characteristics of the filler material 152 can be accomplished at temperatures that will not damage the sensitive electrical components. Additionally, in the process of cooling the filler material 152, the blocks of the SEBS copolymer separate to form a physically crosslinked, reversible gel, such that the gel can be reformed to its flowable state if desired. For example, circumstances may occur whereby a failure analysis on the electrical components of the implantable medical device 100 is desired. The reversibility of the gel filler material 152 facilitates removal of the filler material for such analysis, which would not be possible in devices backfilled with a rigid, solid material such as a cured epoxy.

Without limiting the scope of suitable filler materials 152 to any specific formulation, examples of materials that can be used as the filler material 152 can include the SEBS ballistic polymer marketed under the Perma-Gel™ brand by Perma-Gel, Inc. of Albany, OR. Another exemplary material suitable for use as the filler material 152 is the thermoplastic gel marketed under the C-Flex® brand by Saint-Gobain Performance Plastics blended with a mineral oil or comparable plasticizing oil, such as is described in U.S. Pat. No. 4,613,640 which is incorporated herein by reference in its entirety.

When disposed in the residual volume 153 of the housing 102 and encapsulating the electrical components located within the housing 102, the filler material 152 functions to support the electrical components and effectively dampen mechanical stresses and vibrations the electrical components may experience prior to use (e.g., during packaging, shipping and handling prior to implantation). The dampening/support function of the filler material 152 also advantageously minimizes stresses on individual components contained within the housing 102 during use. As such, medical devices in accordance with various embodiments herein can exhibit enhanced durability with respect to forces stemming from pressure changes, shock, vibration, and the like. This is a particularly important benefit in the context of implantable medical devices implanted subpectorally because of the applied forces associated with muscle contraction and breathing.

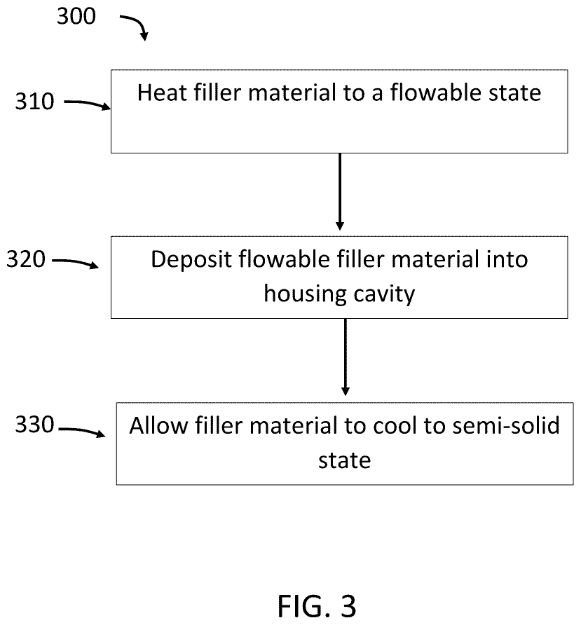

FIG. 3 is a flow chart illustrating an exemplary method 300 of manufacturing an implantable medical device having electrical components disposed within a cavity of a housing, according to an embodiment of the present disclosure. As shown, at block 310, the method 300 includes heating a gel filler material to a liquid, flowable state. In embodiments, the gel filler material can comprise any of the aforementioned thermoplastic gel materials. In an embodiment, the gel filler material can be heated to a temperature above 70 degrees Celsius, which is sufficiently high to liquify the material but well below the temperature at which the electrical components of the implantable medical device would be damaged or otherwise adversely affected.

At block 320, the method 300 includes depositing the gel filler material in the liquid, flowable state into the cavity to substantially encapsulate the electrical components within the gel filler material.

The liquified filler material can be deposited by various techniques. In an embodiment, the housing of the implantable medical device may include openings or ports providing access to the internal cavity, and the flowable fill material can be introduced through one or more of these ports functioning as fill ports, while gas can be exhausted through one or more other ports that function as exhaust ports as the residual volume within the cavity is filled with the filler material. The port location(s) for each device can be selected to avoid potential contact with internal electronic components and to optimize the encapsulation of the internal electrical components as well as removal of any residual gas from the cavity of the housing.

In one embodiment, the filler material can be introduced into cavity by injecting the liquid filler material through the fill port(s) using a syringe or similar instrument. In other embodiments, the housing can be submerged in a bath of the liquid filler material.

At block 330, the method 300 includes allowing the gel filler material to cool to a semi-solid state. The step of allowing the gel filler material to cool can include passively allowing filler material to cool, for example, by placing the filled housing in an environment having an ambient temperature below the temperature at which the filler material transitions from its flowable, liquid state to its semi-solid state. Alternatively, the filler material can be actively cooled in a refrigerated environment.

In embodiments where ports are formed in the housing, the ports can be plugged or sealed after the liquid composition is deposited. As an example, the ports can be sealed by welding a metallic (e.g., titanium where the housing is formed therefrom) patch over them.

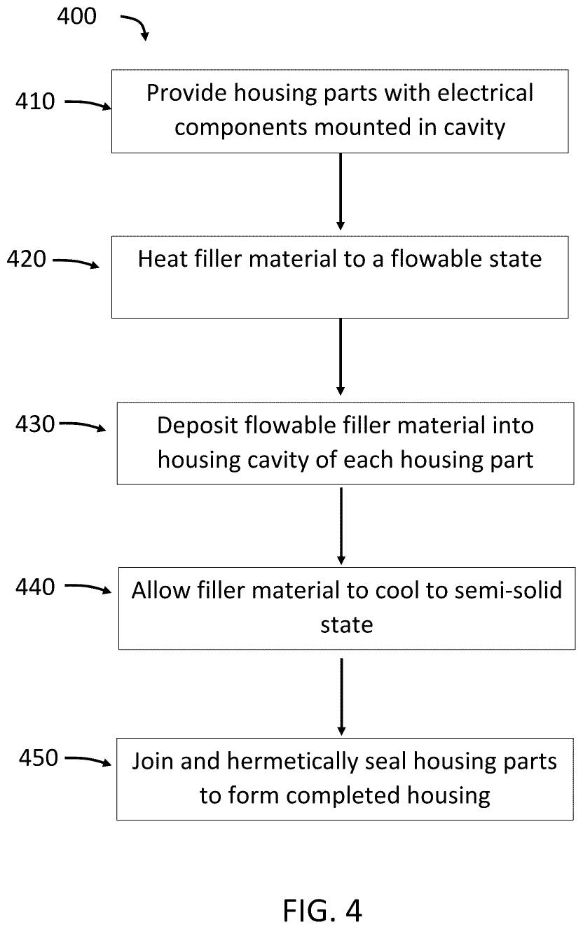

In other embodiments, the filler material can be deposited before the housing is fully formed. For example, FIG. 4 is a flow chart illustrating an alternative method 400 of manufacturing an implantable medical device having electrical components disposed within a cavity of a housing, according to an embodiment of the present disclosure. As shown, at block 410, separate housing parts that each define a cavity portion are provided, and a set of electrical components is mounted into each respective housing part. At block 420, the method 400 includes heating a gel filler material to a liquid, flowable state, as also described above with respect to the method 300. Then, at block 430, the flowable filler material can be deposited into each respective housing part cavity to encapsulate the electrical components therewithin. At block 440, the filler material is allowed to cool to a semi-solid state, again in the same manner described in connection with the method 300. Finally at block 450, the housing parts can subsequently joined and the housing hermetically sealed after the filler material cools to its semi-solid state.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112 (f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A method of manufacturing an implantable medical device having electrical components disposed within a cavity of a housing, the method comprising:
heating a gel filler material to a liquid, flowable state, wherein the gel filler material comprises a styrene-ethylene-butylene-styrene (SEBS) thermoplastic block copolymer and a plasticizing oil;
depositing the gel filler material in the liquid, flowable state into the cavity to substantially encapsulate the electrical components within the gel filler material; and
allowing the gel filler material to cool to a semi-solid state.

2. The method of claim 1, wherein the housing comprises a first housing part and the cavity comprises a first cavity portion defined by the first housing part, and wherein a first set of the electrical components is disposed within the first cavity portion, and wherein depositing the gel filler material includes depositing the gel filler material in the first cavity portion to substantially encapsulate the first set of the electrical components.

3. The method of claim 2, wherein the housing comprises a second housing part and the cavity comprises a second cavity portion defined by the second housing part, and wherein a second set of the electrical components is disposed within the second cavity portion, and wherein depositing the gel filler material includes depositing the gel filler material in the second cavity portion to substantially encapsulate the second set of the electrical components.

4. The method of claim 3, further comprising joining the first and second housing parts after allowing the gel filler material to cool to the substantially semi-solid state.

5. The method of claim 1, further comprising:
prior to depositing the gel filler material, forming the housing by joining first and second housing parts to define the cavity with the electrical components disposed therein, wherein the housing includes a fill port and an exhaust port; and wherein depositing the gel filler material includes introducing the gel filler material into the cavity through the fill port; and
hermetically sealing the fill port and the exhaust port after allowing the gel filler material to cool to the substantially solid state.

6. The method of claim 1, wherein heating the gel filler material includes heating the gel filler material to a temperature above 70 degrees C.

7. The method of claim 6, wherein allowing the gel filler material to cool includes allowing the gel filler to cool to a temperature below 70 degrees C.

8. The method of claim 6, wherein allowing the gel filler material to cool includes actively cooling the gel filler material in an environment below ambient temperature.

9. The method of claim 1, wherein the plasticizing oil is a mineral oil.

10. An implantable medical device, comprising:
a housing defining a hermetically sealed cavity;
electrical components mounted within the housing; and
a semi-solid gel filler material disposed within the cavity and substantially encapsulating the electrical components, the gel filler material comprising a thermoplastic block copolymer.

11. The implantable medical device of claim 10, wherein the thermoplastic block copolymer is a styrene-ethylene-butylene-styrene (SEBS) thermoplastic block copolymer.

12. The implantable medical device of claim 11, wherein the gel filler material further comprises a plasticizing oil.

13. The implantable medical device of claim 12, wherein the plasticizing oil is a mineral oil.

14. The implantable medical device of claim 13, wherein the housing comprises a first housing part and the cavity comprises a first cavity portion defined by the first housing part, and wherein a first set of the electrical components is disposed within the first cavity portion, and wherein the semi-solid gel filler material is disposed in the first cavity portion to substantially encapsulate the first set of the electrical components.

15. The implantable medical device of claim 14, wherein the housing comprises a second housing part and the cavity comprises a second cavity portion defined by the second housing part, and wherein a second set of the electrical components is disposed within the second cavity portion, and wherein the semi-solid gel filler material is disposed in the second cavity portion to substantially encapsulate the second set of the electrical components.

16. The implantable medical device of claim 15, wherein the first housing part and the second housing part are joined to form the housing.

17. An implantable medical device comprising:
a hermetically sealed housing forming an internal cavity;
electrical components mounted within the cavity of the housing, wherein portions of the cavity not occupied by the electrical components define a residual volume of the housing; and
a semi-solid gel filler material filling the residual volume and substantially encapsulating the electrical components, the gel filler material assuming a flowable liquid state at a temperature of 70 degrees Celsius or greater, and capable of transitioning to semi-solid state at a temperature below 70 degrees Celsius.

18. The implantable medical device of claim 17, wherein the semi-solid gel filler material comprises a thermoplastic block copolymer and a plasticizing oil.

19. The implantable medical device of claim 18, wherein the thermoplastic block copolymer is a styrene-ethylene-butylene-styrene (SEBS) thermoplastic block copolymer.

20. The implantable medical device of claim 19, wherein the plasticizing oil is a mineral oil.
